(19) United States
(12) Patent Application Publication
(10) Pub. No.: US 2025/0116601 A1
(43) Pub. Date: Apr. 10, 2025

(54) SPECTROSCOPIC GAS SENSING WITH LOCALITY-SENSITIVE HASHING OF MEASURED SPECTRA

(71) Applicant: THE REGENTS OF THE UNIVERSITY OF COLORADO, A BODY CORPORATE, Denver, CO (US)

(72) Inventors: Gregory B. RIEKER, Boulder, CO (US); Charles Scott CALLAHAN, Boulder, CO (US)

(21) Appl. No.: 18/730,240

(22) PCT Filed: Jan. 20, 2023
(86) PCT No.: PCT/US2023/011240

§ 371 Date: Jul. 18, 2024

Related U.S. Application Data
(60) Provisional application No. 63/266,946, filed on Jan. 20, 2022.

(51) Int. Cl. G01N 21/39; G01N 33/00

(52) U.S. Cl. CPC G01N 21/39; G01N 33/0027; G01N 2021/399

(57) ABSTRACT

A method for spectroscopic gas sensing processes a measured spectrum generated by an optical spectrometer. The method includes transforming, with a locality-sensitive hash function, the measured spectrum into an integer hash value h. The method also includes adding, to a candidate set of candidate spectra, template spectra stored in an h^{th} bin of a look-up table. The method also includes calculating, based on each candidate spectrum in the candidate set, a measure that quantifies discrepancy between the measured spectrum and said each candidate spectrum. The method also includes identifying, based on the measure, a best-match spectrum of the candidate spectra, and retrieving, from the h^{th} bin of the look-up table, a parameter set corresponding to the best-match spectrum. The method also includes deriving, based on the parameter set, one or more properties of the gas sample.

### RELATED APPLICATIONS

This application claims priority to U.S. Provisional Patent Application No. 63/266,946, filed on Jan. 20, 2022, the entirety of which is incorporated herein by reference.

### BACKGROUND

Many gas sensors use spectroscopy to measure or infer physical properties of a gas sample. Examples of such physical properties include concentration, temperature, and pressure. These spectroscopic gas sensors use various signal processing techniques to convert raw spectroscopic data into estimates of the physical properties.

### SUMMARY

One technique that can be used to derive physical properties from raw spectroscopic data is fitting a measured spectrum to a mathematical model. For example, non-linear least-squares can be implemented with a Levenberg-Marquardt algorithm to identify a set of best-fit parameters that optimize agreement between the model and the measured spectrum. These best-fit parameters can then be processed to calculate the physical properties. Other nonlinear regression algorithms and techniques may be used

Nonlinear regression suffers from several drawbacks. First, it is computationally intensive because it typically involves many operations, like division, that are relatively time-consuming and inefficient. Second, many regression algorithms are iterative, continuing until some level of convergence has been reached. However, these algorithms sometimes do not converge, such as when the initial values of the parameters are not well selected. It can also be challenging to identify initial values with sufficient accuracy to ensure convergence. Third, the number of iterations needed to reach convergence can vary depending on the initial values. Accordingly, the algorithm may not always have the same execution time, which can lead to back-logs of data and delays in operation of the gas sensor.

To avoid these drawbacks, regression can be avoided altogether. For example, the measured spectrum can be compared to several “template” spectra that are stored in, and retrieved from, a look-up table. Each template spectrum is generated from the mathematical model using one unique set of values for the parameters. Calculating the discrepancy between the measured spectrum and the template spectrum (e.g., a residual sum of squares) can be performed quickly and efficiently (e.g., using a microprocessor). The template spectrum that gives rise to the smallest discrepancy may then be selected as a best-fit spectrum. The corresponding parameters used to generate the best-fit spectrum approximate the best-fit parameters that would have been obtained with non-linear regression. These parameters may then be processed to derive the properties of the sample.

In a simple implementation of a look-up table, the measured spectrum is compared to every template spectrum in the look-up table. The time complexity for this operation is O(N), where N is the number of template spectra in the look-up table. Although such linear scaling is generally considered efficient, N may be so large that this comparison becomes too time-consuming to be practical (e.g., several seconds, or longer). For example, the look-up table could store millions of template spectra, or more.

One way to avoid having to access every template spectrum in the look-up table is to implement the look-up table as a hash table. Here, the measured spectrum can be fed into a hash function that returns an integer hash value h. The template spectrum and parameters stored in the h^{th} bin, or bucket, of the hash table are retrieved. These parameters may then be used as the best-fit parameters for deriving the physical properties of the gas sample. Advantageously, the use of a hash table dramatically improves time complexity to O(1).

One drawback to hash tables is that the resulting hash values are overly sensitive to noise in the measured spectrum. Specifically, a measured spectrum may be decomposed into a signal component and a noise component. Two measured spectra with the same signal component but different noise components should produce the same hash value (i.e., a collision), thereby leading to the same set of parameters stored in the same bin of the hash table. However, conventional hash functions are designed to avoid collisions between inputs that are not exactly identical. Accordingly, these two spectra will yield different hash values, and therefore two different sets of parameters stored in two different bins of the hash table.

The present embodiments solve this problem with a look-up table whose bins are accessed using a locality sensitive hash function. Locality sensitive hashing is designed to maximize the probability of a collision between two inputs that are similar, but not identical. In this case, each bin of the look-up table may store several template spectra and corresponding parameters. The time complexity of accessing one bin of the look-up table advantageously remains O(1). As described in more detail below, additional processing is needed to determine which of the template spectra in the one accessed bin best matches the measured waveform. However, this additional processing can be performed quickly.

The present embodiments include spectroscopic gas sensors that utilize locality sensitive hashing to vastly speed up signal processing of raw spectra obtained from an optical spectrometer. Advantageously, the present embodiments may be implemented with a host of spectroscopy techniques known in the art, including wavelength modulation spectroscopy, frequency modulation spectroscopy, and Doppler-free absorption spectroscopy. The present embodiments can operate at rates exceeding 1 kHz.

One application of the present embodiments is reducing emissions and increasing efficiency of natural-gas-powered engines. Heavy-duty trucks and marine vessels operating on diesel and gasoline account for 7% of US energy consumption, a significant, addressable source of greenhouse gas (GHG) emissions. While GHG-reducing electrification is rising among light-duty vehicles, there remain challenges to electrifying long-haul heavy-duty vehicles due to demanding load and mileage requirements. Natural gas as fuel may help reduce GHG production. While natural gas can offer up to a 25% reduction in GHG emission over diesel fuel, reaching parity with diesel efficiency while meeting evolving modern emissions regulations is challenging and has slowed proliferation of this technology.

One strategy to reduce emissions and increase efficiency in commercial natural gas-powered engines is to precisely control the air-to-fuel ratio and exhaust gas recirculation (EGR) rates to maximize power density. This strategy can be implemented with a closed-loop control system coupled with fast gas sensors. The spectroscopic gas sensors herein are fast enough to use in such a closed-loop system, and therefore could be used as part of a high-efficiency natural-gas-powered engine.

### BRIEF DESCRIPTION OF THE FIGURES

F FIG. 1 is a block diagram of a spectroscopic gas sensor that uses locality-sensitive hashing of a measured spectrum to determine one or more properties of a gas sample, in embodiments.

FIG. 2 is a block diagram of a spectroscopic gas sensor that combines the line-shape processor of FIG. 1 with an optical spectrometer that implements single-pass absorption spectroscopy with an unmodulated laser beam, in embodiments.

FIG. 3 is a block diagram of a spectroscopic gas sensor that combines the line-shape processor of FIG. 1 with an optical spectrometer that implements wavelength modulation spectroscopy, in embodiment.

FIG. 4 is a block diagram of a spectroscopic gas sensor that combines the line-shape processor of FIG. 1 with an optical spectrometer that implements calibration-free wavelength modulation spectroscopy, in embodiments.

FIG. 5 illustrates a method that uses locality sensitive hashing to determine the properties of the gas sample.

FIG. 6 shows the structure of a look-up table, in an embodiment.

FIG. 7 shows one block that is formed by segmenting and concatenating a first in-phase signal, a first quadrature signal, a second in-phase signal, and a second quadrature signal, in an embodiment.

FIG. 8 illustrates a method for constructing the look-up table of FIG. 6, in embodiments.

FIG. 9 shows a theoretical noise-free spectrum and a noisy spectrum generated by adding noise to the noise-free spectrum.

FIG. 10 is a plot showing averaged bin-number error as a function of noise level.

### DETAILED DESCRIPTION

FIG. 1 is a block diagram of a spectroscopic gas sensor 100 that uses locality-sensitive hashing of a measured spectrum 102 to determine one or more properties 108 of a gas sample 106. An optical spectrometer 104 generates the measured spectrum 102 by probing the gas sample 106 with a laser beam 116. The optical spectrometer 104 includes a laser 110 that generates the laser beam 116 and a controller 112 that controls the laser 110 to vary the frequency f_{L} (or, equivalently, the wavelength λ_{L}) of the laser beam 116. The optical spectrometer 104 also includes a photodetector 120 that detects the laser beam 116 after transmission through the gas sample 106. The optical spectrometer 104 also includes a signal processor 114 that processes an output 138 of the photodetector 120.

The measured spectrum 102 is a set of n spectroscopic values {α₁, α₂ . . . , αₙ}. Each spectroscopic value αᵢ (1≤i≤n) is measured at a corresponding frequency fᵢ of the laser beam 116. Each spectroscopic value αᵢ may be a singular measurement of absorption or dispersion (i.e., phase shift), or a combination of such measurements. The n spectroscopic values may be stored in memory as an array S of n elements in which the i^{th} array element S[i] stores the i^{th} spectroscopic value αᵢ. When the i^{th} frequency fᵢ can be derived from the index i, it does need not to be stored in the array S. The n spectroscopic values may be stored in the array S as a sequence ordered by the n frequencies f₁, f₂, . . . fₙ. The frequencies f₁, f₂, . . . fₙ may be uniformly spaced in frequency, although this is not necessary. Alternatively, the array may be ordered by the n wavelengths λ₁, λ₂, . . . λₙ. The wavelengths λ₁, λ₂, . . . λₙ may be uniformly spaced, although this is not necessary.

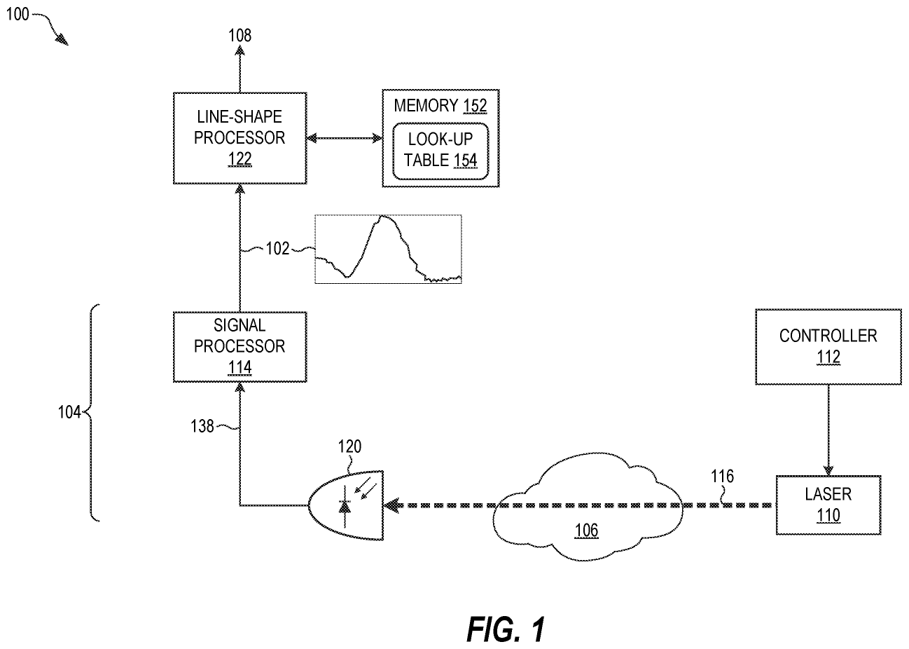

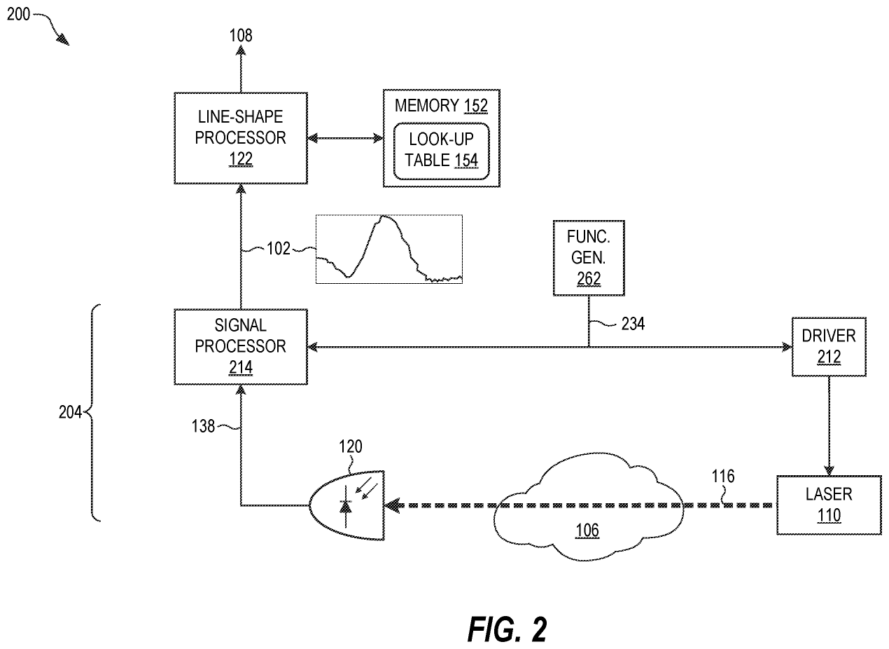

As an example of how the i^{th} frequency fᵢ can be derived from the index i, the i^{th} spectroscopic value αᵢ may have a corresponding time value tᵢ indicating when the spectroscopic value αᵢ was measured. In this case, the frequency fᵢ can be derived from the time value tᵢ (e.g., based on a predetermined frequency schedule). The n spectroscopic values may be stored in the array S as a sequence ordered by the n time values t₁, t₂, . . . , tₙ. In this case, the array S may be thought of as a time series. The time values t₁, t₂, . . . , tₙ may be uniformly spaced in time, although this is not necessary. Sweeping the laser frequency f_{L} with a ramp signal (see signal 234 in FIGS. 2-4) is one example of a frequency schedule that creates an association between laser frequency f_{L} and time.

The measured spectrum 102 may additionally store the frequencies f₁, f₂, . . . fₙ, the time values t₁, t₂, . . . , tₙ, additional data, or any combination thereof. In these cases, the measured spectrum 102 is a set of tuples {a₁, a₂ . . . , aₙ} in which the i^{th} tuple aᵢ contains two or more entries. For example, the i^{th} tuple aᵢ may be aᵢ=(aᵢ, fᵢ, . . . ), where the first entry is the spectroscopic value di and the second entry is the frequency fᵢ. The tuples may have alternative or additional entries without departing from the scope hereof. The tuples may be stored as a sequence ordered by any of their entries.

The spectroscopic gas sensor 100 includes a line-shape processor 122 that processes the measured spectrum 102 to determine and output the one or more properties 108. The line-shape processor 122 compares the measured spectrum 102 to a set of candidate spectra that are retrieved from a look-up table 154 storing a plurality of template spectra. The look-up table 154 may have thousands of bins (also referred to as buckets) or more, each storing one or more template spectra and corresponding parameters. In this case, the look-up table 154, due to its large size, may be stored in an external memory (e.g., a memory card or hard drive). More details about the look-up table 154 and line-shape processor 122 are presented below. In some embodiments, the gas sensor 100 excludes the optical spectrometer 104. For example, the optical spectrometer 104 may be operated or provided by a third party that sends the measured spectrum 102 to the line-shape processor 122 (e.g., via a computer network).

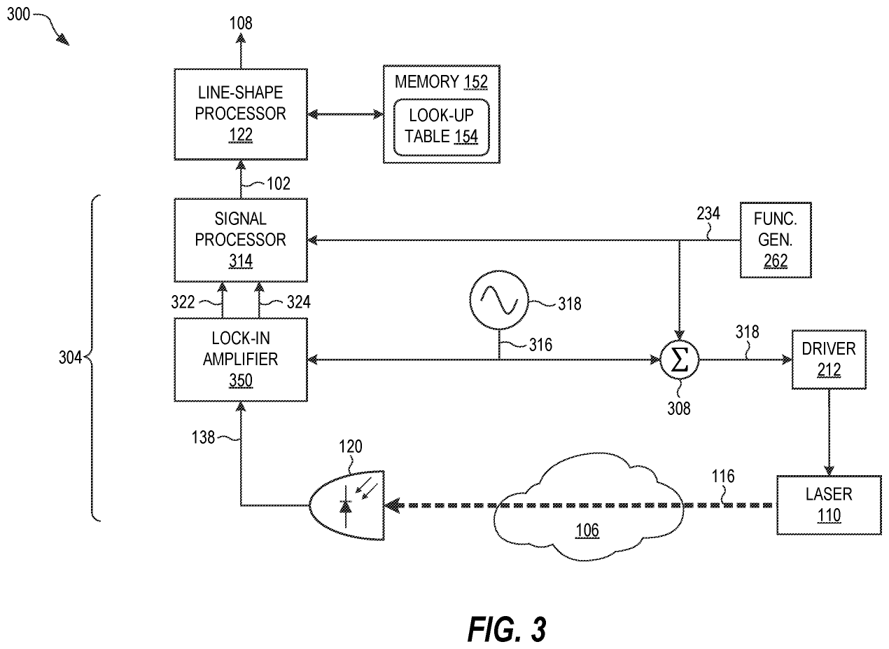

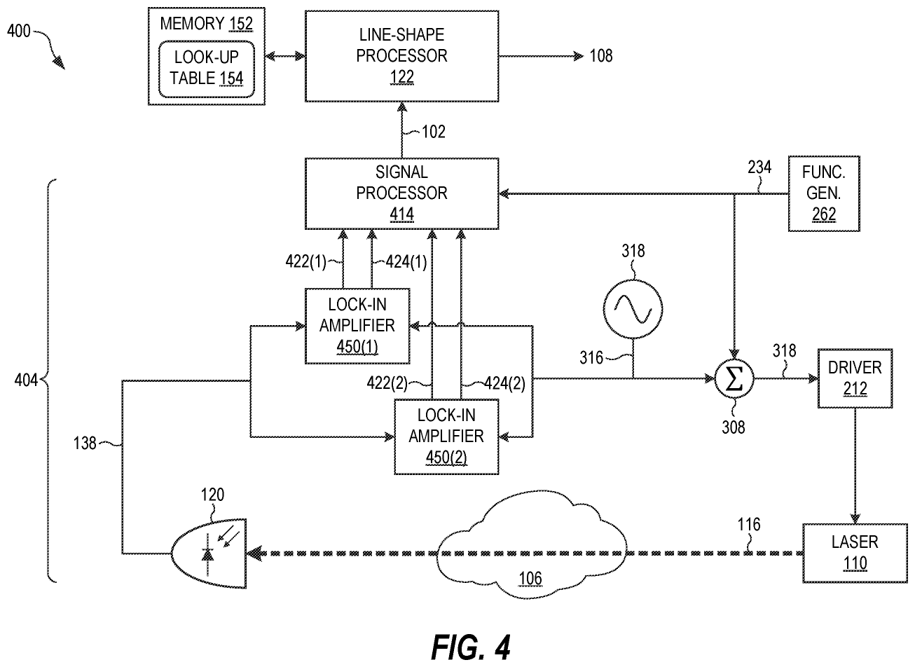

The optical spectrometer 104 may implement any type of spectroscopy that can generate the measured spectrum 102. FIGS. 2-4 illustrate various types of spectrometry that can be used with the present embodiments. Specifically, FIG. 2 shows absorption spectroscopy in which the frequency f_{L} of the laser beam 116 is swept, but otherwise unmodulated. FIG. 3 shows wavelength modulation spectroscopy in which the frequency f_{L} is swept and modulated. FIG. 4 shows calibration-free wavelength modulation spectroscopy that corrects for residual amplitude modulation that is introduced when frequency-modulating the laser 110.

FIG. 2 is a block diagram of a spectroscopic gas sensor 200 that combines the line-shape processor 122 of FIG. 1 with an optical spectrometer 204 that implements single-pass absorption spectroscopy with an unmodulated laser beam. The optical spectrometer 204 is an example of the optical spectrometer 104 of FIG. 1 that includes a function generator 262 for generating a ramp signal 234. In response to the ramp signal 234, the driver 212 controls the laser 110 to sweep the frequency f_{L} of the laser beam 116. If the ramp signal 234 is a triangle wave or sawtooth wave, the frequency f_{L} will be swept linearly. The optical spectrometer 204 also includes a signal processor 214 that is an example of the signal processor 114 of FIG. 1. The signal processor 214 uses the ramp signal 234 to process the output 138 synchronously with the frequency f_{L}, as swept, to generate the measured spectrum 102.

The laser 110 may be a tunable diode laser. In this case, the driver 212 may be a current source that is modulated by the ramp signal 234. Examples of tunable diode lasers include, but are not limited to, a Fabry-Perot laser diode, a distributed feedback (DFB) laser, a distributed Bragg reflector (DBR) laser, and a vertical cavity surface emitting laser (VCSEL). The VCSEL may be a micro-electromechanical system VCSEL (MEMS-VCSEL). The laser 110 may alternatively be an external-cavity laser, in which case the frequency sweep can be implemented by modulating the length of the external cavity. The laser 110 may alternatively be a fixed-frequency laser, in which case the frequency f_{L} can be adjusted using a phase or frequency modulator (e.g., an electro-optic modulator or acousto-optic modulator).

Other techniques for controlling the frequency f_{L} that are known in the art may be used with the present embodiments. For example, the optical spectrometer 204 may include a reference laser that is locked to an optical frequency reference (e.g., a molecular or atomic transition, a resonance of a high-finesse Fabry-Perot cavity, etc.). The laser 110 may be offset phase-locked to the reference laser. Changing the offset frequency of the phase-lock loop changes the frequency f_{L} relative to that of the reference laser. If the offset frequency is changed linearly in time (e.g., one period of a triangle or sawtooth wave), the frequency f_{L} will similarly be swept linearly in time. However, the offset frequency can be varied in time in a different manner (e.g., randomly in time, linearly in corresponding wavelength, quadratically, etc.) without departing from the scope hereof.

Another technique for controlling the frequency f_{L} is to offset phase-lock the laser 110 to a tooth of an optical frequency comb. The optical frequency comb may be stabilized to an optical or microwave frequency reference, thereby transferring the frequency stability of the reference to the frequency of the tooth. Changing the offset frequency of the phase-lock loop changes the frequency f_{L} relative to that of the comb. The frequency f_{L} can also be changed by adjusting one or both of the comb offset and comb repetition rate of the optical frequency comb. In this case, the offset frequency between the laser 110 and the tooth may be kept fixed.

The absorption spectroscopy implemented by the optical spectrometer 204 is relatively simple since there is no modulation and demodulation. In this case, the optical spectrometer 204 can alternatively be constructed using a tunable incoherent light beam instead of the laser beam 116. Such an incoherent light beam can be generated with a tunable incoherent light source, such as a lamp with a broadband output that is filtered with a tunable filter (e.g., a monochromator or similar type of grating-based optical filter). Accordingly, it should be recognized that many of the present embodiments may be implemented with incoherent light by replacing the laser 110 with a tunable source of incoherent light.

FIG. 3 is a block diagram of a spectroscopic gas sensor 300 that combines the line-shape processor 122 of FIG. 1 with an optical spectrometer 304 that implements wavelength modulation spectroscopy. The optical spectrometer 304 is an example of the optical spectrometer 204 of FIG. 2 that further includes an oscillator 318 for generating a modulation signal 316. The modulation signal 316 is a sine wave having a modulation frequency fₘ and a modulation amplitude Aₘ. An adder 308 sums the modulation signal 316 and the ramp signal 234 into a combined signal 318 that simultaneously sweeps and frequency modulates the laser 110. The ramp signal 234 has a ramp frequency fᵣ and a ramp amplitude Aᵣ. It is assumed that the ramp frequency fᵣ is less than the modulation frequency fₘ. The ramp amplitude Aᵣ is selected so that the frequency f of the laser beam 116 is swept partially or entirely over an absorption feature of interest of the gas sample 106.

The optical spectrometer 304 also includes a lock-in amplifier 350 that uses the modulation signal 316 as a reference signal for demodulating the output 138 of the photodetector 120. The lock-in amplifier 350 may demodulate at the modulation frequency fₘ or a harmonic thereof (e.g., 2fₘ, 3fₘ, etc.). The lock-in amplifier 350 outputs an in-phase signal 322 and a quadrature signal 324 that a signal processor 314 processes to generate the measured spectrum 102. The signal processor 314 is an example of the signal processor 214 of FIG. 2. The signal processor 314 may also normalize the measured spectrum 102 (e.g., using one or both of the in-phase signal 322 and quadrature signal 324).

Without the ramp signal 234 (e.g., Aᵣ is set to 0), the in-phase signal 322 and quadrature signal 324 would be constant in time (ignoring noise and drift of the laser frequency f_{L}). With the ramp signal 234, the in-phase signal 322 and quadrature signal 324 both vary synchronously with the ramp signal 234. The signals 322 and 324 therefore form scans, over frequency, of the absorption feature, with each of the signals 322 and 324 containing different information (i.e., absorption and dispersion) about the absorption feature.

In some embodiments, the spectroscopic gas sensor 300 includes multiple lasers that are modulated at different modulation frequencies. These lasers may have different wavelengths so that they simultaneously interact with different absorption features of the gas sample 106. The outputs of the lasers may be combined into a single laser beam that, after transmission through the sample 106, is detected by the photodetector 120. The output 138 is then demodulated by at least one lock-in amplifier for each modulation frequency, thereby allowing spectroscopic values to be measured for each laser (i.e., each absorption feature).

FIG. 4 is a block diagram of a spectroscopic gas sensor 400 that combines the line-shape processor 122 of FIG. 1 with an optical spectrometer 404 that implements calibration-free wavelength modulation spectroscopy. More details about calibration-free wavelength modulation spectroscopy can be found in Gregory B. Rieker, Jay B. Jeffries, and Ronald K. Hanson, “Calibration-free wavelength-modulation spectroscopy for measurements of gas temperature and concentration in harsh environments,” Appl. Opt. 48, 5546-5560 (2009).

The optical spectrometer 404 is an example of the optical spectrometer 304 of FIG. 3 that includes an additional lock-in amplifier for demodulating the output 138 of the photodetector 120. Specifically, the optical spectrometer 304 includes a first lock-in amplifier 450(1) that demodulates the output 138 synchronously with the modulation signal 316 to generate a first in-phase signal 422(1) and a first quadrature signal 424(1). The optical spectrometer 304 also includes a second lock-in amplifier 450(2) that demodulates the output 138 synchronously with the modulation signal 316 to generate a second in-phase signal 422(2) and a second quadrature signal 424(2).

The lock-in amplifiers 450(1) and 450(2) demodulate the output 138 at different harmonics of the modulation signal 316. Specifically, the first lock-in amplifier 450(1) is configured to demodulate at the j^{th} harmonic jfₘ while the second lock-in amplifier 450(2) is configured to demodulate at the k^{th} harmonic kfₘ, where j≠k. In one example, j=1 and k=2, i.e., the first lock-in amplifier 450(1) operates at the first harmonic 1fₘ and the second lock-in amplifier 450(2) operates at the second harmonic 2fₘ. In another example, j=2 and k=3. The integers j and k may have other values without departing from the scope hereof.

The optical spectrometer 404 also includes a signal processor 414 that is an example of the signal processor 314 of FIG. 3. The signal processor 414 processes the first in-phase signal 422(1), the first quadrature signal 424(1), the second in-phase signal 422(2), and the second quadrature signal 424(2) to generate the measured spectrum 102. In particular, the signal processor 414 may (i) process the signals 422(1) and 424(1) to generate an unnormalized spectrum, (ii) process the signals 422(2) and 424(2) to generate a normalization spectrum, and (iii) divide the unnormalized spectrum by the normalization spectrum to generate the measured spectrum 102. More details about this normalization procedure are described below (e.g., see Eqn. 1) and in the above-mentioned reference by Gregory B. Rieker et al.

In FIGS. 1-4, several of the electronic components may be implemented as digital circuits. Such components include the signal processor 114, the lock-in amplifier 350, the oscillator 318, the function generator 262, and the adder 308. For example, where a lock-in amplifier (e.g., the lock-in amplifiers 450(1) and 450(2) in FIG. 4) is implemented digitally, an analog-to-digital converter may be used to digitize the analog output 138 of the photodetector 120. The digital lock-in amplifier may demodulate the resulting digital signal by multiplying it with a digital local-oscillator waveform to generate a digital in-phase waveform and a digital quadrature waveform. The signal processor 114 may then digitally process the digital in-phase and quadrature waveforms to generate the measured spectrum 102 as data (e.g., an array stored in memory). In another example, one or more of the function generator 262, oscillator 318, and adder 308 are implemented digitally. In this case, a digital-to-analog converter may be used to convert the (digital) combined signal 318 into an analog signal that controls the driver 212. The oscillator 318 may be digitally implemented, for example, using direct digital synthesis.

In embodiments, the line-shape processor 122 is implemented as a digital circuit. In some of these embodiments, the line-shape processor 122 may advantageously be implemented using the same digital circuit as other components. For example, in FIG. 4 the lock-in amplifiers 450(1) and 450(2), oscillator 318, function generator 262, adder 308, signal processor 414, and line-shape processor 122 may all be implemented using an integrated circuit (e.g., a field-programmable gate array (FPGA), digital signal process (DSP) chip, central processing unit (CPU), graphics processing unit (GPU), microcontroller, etc.). The integrated circuit may be a system-on-chip (SoC) that combines one or more CPU cores with one or more digital signal processing cores. The system-on-chip may alternatively combine the one or more CPU cores with programmable logic (i.e., an FPGA) that is pre-programmed to implement at least some of the signal-processing functionality described herein.

Those trained in the art will recognize that it is not necessary for lock-in amplifiers, function generators, oscillators, and other components of the present embodiments to be implemented digitally. Accordingly, the present embodiments include any combination of digital and analog implementations of these components. For example, the lock-in amplifiers 450(1) and 450(2) can be implemented as analog circuits while the signal processor 122 is digital. In this case, analog-to-digital converters may be used to digitize the signals 422(1), 424(1), 422(2), and 424(2) for subsequent processing by the signal processor 122.

The gas sample 106 need not be physically confined. For example, the gas sample 106 could be air or gas in the atmosphere. Alternatively, the gas sample 106 can be confined in a chamber. The chamber may be a vapor cell (e.g., made of optically transparent glass or sapphire) or a stainless-steel vacuum system with optical viewports. In any case, the chamber may include a first window through which light from the tunable laser 110 enters the chamber. The chamber may also include a second window through which the light, after passing through the gas sample 106, exists the chamber to reach the photodetector 120.

In FIG. 1, the sample 106 is shown as a cloud of gas. However, the sample 106 may be any type of matter that can be spectroscopically measured, including liquids (e.g., biological fluids, water, etc.), solids (e.g., non-linear optical elements, semiconductor materials, etc.), and plasmas. While the embodiments described above focus on spectroscopy techniques based on single-pass transmission of a laser beam through the gas sample 106, other spectroscopy techniques can be used to generate the measured spectrum 102. Examples include, but are not limited to, Doppler-free spectroscopy that uses counterpropagating and overlapped pump and probe laser beams (e.g., derivative spectroscopy, frequency-modulation spectroscopy, modulation-transfer spectroscopy, etc.), multi-pass spectroscopy, dispersive spectroscopy, Fourier-transform infrared spectroscopy, dual comb spectroscopy, direct frequency-comb spectroscopy, cavity-enhanced spectroscopy, grating spectroscopy, tunable laser absorption spectroscopy, cavity ring-down spectroscopy, hyperspectral imaging, and combinations thereof (e.g., noise-immune cavity-enhanced optical heterodyne spectroscopy). The spectroscopy may be performed in any part of the electromagnetic spectrum (i.e., x-ray, ultraviolet, visible, infrared, terahertz, microwave, etc.).

The gas sample 106 may be located remotely from some or all of the components of the spectroscopic gas sensor 100. For example, the gas sample 106 may be located in a space-limited and environmentally demanding location, such as the exhaust of an engine or inside an industrial furnace. One or more components of the present embodiments (e.g., the line-shape processor 122, the signal processor 114, the lock-in amplifier 350, the function generator 262, the oscillator 318, the driver 212, etc.) may be located away from this location, where the environment is less extreme (e.g., cooler temperatures, less vibration, etc.). In this case, optical fibers and electrical cables may be used, as needed, for transmitting optical and electrical signals, respectively, between those components near the gas sample 106 with those components that are remotely located. Since the laser beam 116 can travel several kilometers, it is possible for all of the components of the gas sensor 100, including the laser 110 and photodetector 120, to be located remotely (e.g., more than 10, 100, or 1000 meters away) from the gas sample 106.

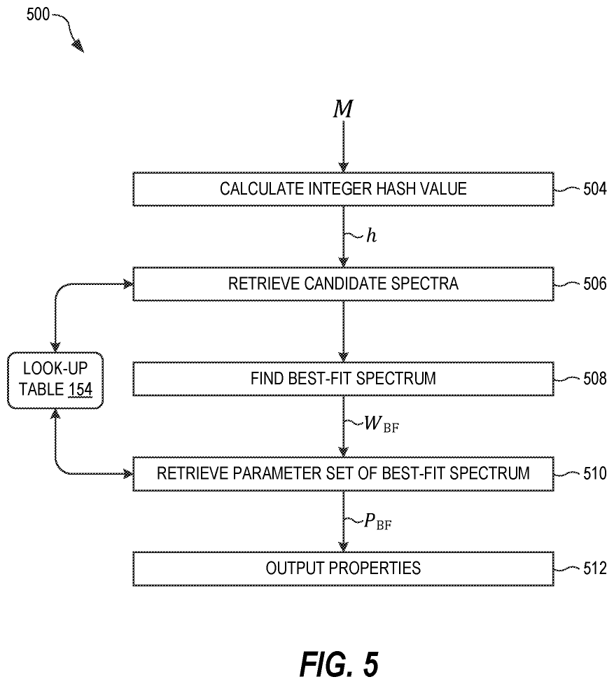

FIG. 5 illustrates a method 500 that uses locality sensitive hashing to determine the properties 108 of the gas sample 106. The method 500 may be performed by the line-shape processor 122 of FIGS. 1-4. The method 500 starts with the measured spectrum 102, which is assumed to be an array M of n elements. In step 504 of the method 500, this measured-spectrum array M is hashed to obtain an integer hash value h. The step 504 uses a locality-sensitive hash function in which two inputs that are similar, but not exactly the same, produce the same hash value with high probability. Locality-sensitive hashing ensures that noise in the array M does not affect the hash value h. For example, consider two arrays M₁ and M₂ whose elements have the same signal content but different noises. These arrays produce integer hash values that are denoted h₁ and h₂, respectively. In a conventional hash function, the hash values h₁ and h₂ are uncorrelated and therefore could be very different from each other. With locality-sensitive hashing, the effect of the different noises is removed so that the hash values h₁ and h₂ are either identical (i.e., h₁=h₂) or nearly so (e.g., |h₁−h₂|=1 or 2). This is equivalent to maximizing the probability of a hash collision when the arrays M₁ and M₂ are similar but not identical.

One aspect of the present embodiments is the realization that locality-sensitive hashing can be efficiently implemented with a measured spectrum by summing the elements of the measured spectrum and truncating one or more of the least-significant digits of the sum. Applying this to the method 500, the elements of the measured-spectrum array M are summed i.e., s=Σᵢ₌₁ⁿM[i]. One or more of the less-significant digits of the sum s are then truncated to obtain the integer hash value h, e.g., h=trunc (s,c)=└10^{c}s┘/10^{c}, where c is the number of digits to truncate and the notation └x┘ indicates the floor of x. Referring to the example above, the arrays M₁ and M₂ have respective sums s₁ and s₂ that would be identical if it were not for their different noises. Truncating some or all of the less-significant digits of the sums s₁ and s₂, whose values are more sensitive to noise, leaves the more-significant digits that are less sensitive to noise. If a sufficient number of less-significant digits are truncated from both the sums s₁ and s₂, then the resulting hash values h₁ and h₂ will be equal.

To convert the hash value h into an integer index for accessing the look-up table 154, the sum s may be divided by a constant to obtain the integral part of the quotient. This may be implemented using the DIV function that is commonly used in many programming languages. Alternatively, the sum s may be divided by the constant to obtain a real-valued quotient that is converted to an integer either by truncating the decimal points (e.g., using a floor function) or rounding. The value of the constant may be selected, based on the expected range of values of the sum s that is expected, such that the resulting values of h span the number of bins in the look-up table 154.

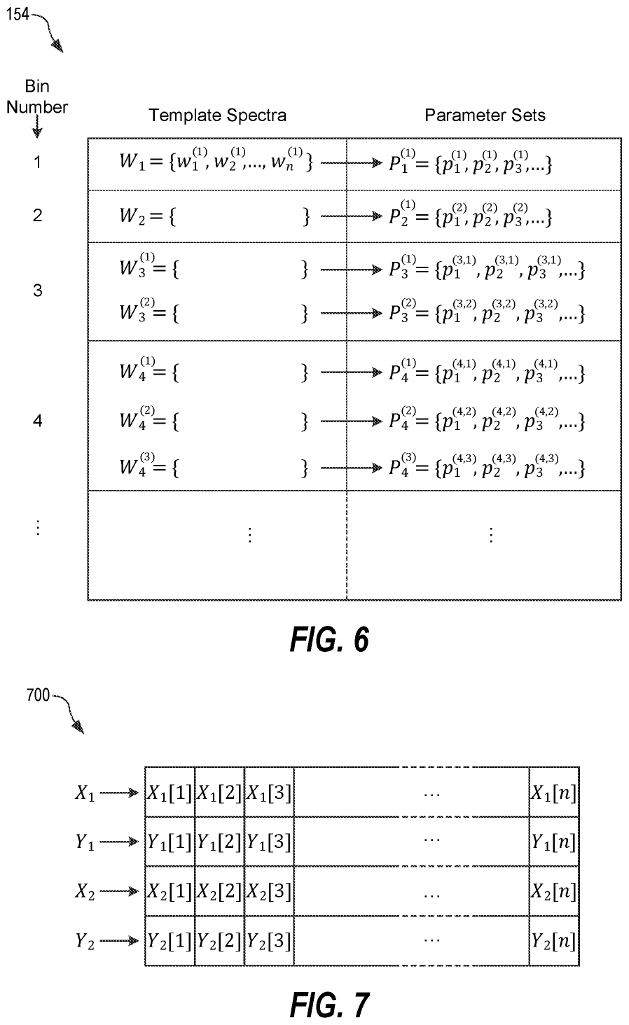

In step 506 of the method 500, the memory 152 is accessed to retrieve template spectra stored in the h^{th} bin, or bucket, of the look-up table 154. The template spectra retrieved from the look-up table 154 form a set of candidate spectra. FIG. 6 shows the structure of the look-up table 154 in more detail. Each bin of the look-up table 154 is shown as a row that is uniquely identified by a bin number. Stored in each bin are one or more template spectra. Also stored in each bin, and in association with each template spectrum, is a parameter set P of one or more parameters {p₁, p₂, . . . }. Each template spectrum is an array of length n, and therefore has the same length as the array M. Although FIG. 6 shows only the first four bins of the look-up table 154, the look-up table 154 may have thousands of bins, or more.

As shown in FIG. 6, some bins of the look-up table 154 may store only one template spectrum and corresponding parameter set. For example, bin 1 has one template spectrum of n elements denoted W₁={w₁⁽¹⁾, w₂⁽¹⁾, w₃⁽¹⁾, . . . , wₙ⁽¹⁾}. The corresponding parameter set is denoted P₁={p₁⁽¹⁾, p₂⁽¹⁾, p₃⁽¹⁾, . . . }. Other bins of the look-up table 154 may store several template spectra and corresponding parameter sets. For example, bin 3 stores two template spectra denoted W₃⁽¹⁾ and W₃⁽²⁾, where the superscripts identify different template spectra within a single bin. For clarity, the elements of W₃⁽¹⁾ and W₃⁽²⁾ are not shown in FIG. 6. The corresponding parameter sets are denoted P₃⁽¹⁾={p₁^{(3,1)}, p₂^{(3,1)}, p³^{^{(3,1)}}, . . . } and P₃⁽²⁾={p₁^{(3,2)}, p₂^{(3,2)}, p₃^{(3,2)}, . . . }. While FIG. 6 shows bin 4 storing three template spectra, any bin in the look-up table 154 may store any number of template spectra (e.g., 10, 100, 1000, or more). How the accuracy and speed of the spectroscopic gas sensor 100 depends on the number of template spectra within each bin is discussed in more detail below.

In step 508 of the method 500, each candidate spectrum W, of the set of candidate spectra, is compared to the measured-spectrum array M to find a best-fit spectrum W_{BF} that best matches M. Each candidate spectrum W may be thought of as a fixed-parameter mathematical model to which the array M can be fitted. A metric may be calculated to quantify the discrepancy between each candidate spectrum W and the array M. For example, the metric may be the residual sum of squares RSS, given mathematically by

$$R S S = {\sum }_{i = 1}^{n} {( W [ i ] - M [ i ] )}^{2} . \qquad ( 3 )$$

In this case, the candidate spectrum W with the lowest RSS is selected as the best-fit spectrum W_{BF}. Other examples of the metric include, but are not limited to, mean square error, mean absolute error, and root mean square error. Some metrics (e.g., mean square error) include division by the number of elements n. Such division is not necessary in embodiments where n does not change. Given that division is a computationally intensive operation, avoiding this division can help conserve computational resources.

In some embodiments, the template spectra stored in one or both of the (h−1)^{th} and (h+1)^{th} bins are also retrieved and added to the set of candidate spectra as part of the step 506. Similarly, the template spectra stored in one or both of the (h−2)^{th} and (h+2)^{th} bins may be retrieved and added to the set of candidate spectra. Retrieving template spectra from more than one bin accounts for the fact that truncation of the sum s may not remove all of the less-significant digits whose values are susceptible to noise. As a result, while there may be a high probability that the best-fit spectrum W_{BF} is one of the template spectra stored in the h^{th} bin, this probability is not unity. If the best-fit spectrum W_{BF} is not stored in the h^{th} bin, then there is a high probability, due to the locality-sensitive hashing, that it is stored in one of the two neighboring (h−1)^{th} and (h+1)^{th} bins, a lower probability that it is stored one of the two next-to-neighboring (h−2)^{th} and (h+2)^{th} bins, and so on. Accordingly, adding, to the set of candidate spectra, the template spectra stored in two or more bins of the look-up table 154 improves the likelihood that the best-fit spectrum W_{BF} is the one template spectrum, of the entire look-up table 154, that best matches the array M.

In step 510 of the method 500, the parameter set P_{BF} of the best-fit spectrum W_{BF} is retrieved. For example, when the look-up table 154 is accessed the first time in the step 506 to retrieve candidate spectra, the parameter set P associated with each template spectrum may be retrieved with the template spectrum. Alternatively, the look-up table 154 may be accessed a second time, after the best-fit spectrum W_{BF} has been identified, to retrieve only the one parameter set P_{BF} of the best-fit spectrum W_{BF}. In step 512, one or more of the parameters of the parameter set P_{BF} are outputted. These parameters may be outputted as the one or more properties 108 of the gas sample 106 in FIG. 1. Alternatively, the one or more properties 108 may be derived from the parameters of the parameter set P_{BF}.

The method 500 may be repeated as the measured-spectrum array M is updated. Repeating the method 500 for a sequence of such arrays M₁, M₂, . . . gives rise to a sequence of the one or more properties 108. Performing the method 500 repeatedly in this manner can be used to identify changes in the one or more properties, from which a change in the gas sample 106 can be inferred. Alternatively, when the gas sample 106 is stable, each of the one or more properties 108 may be averaged over time to reduce statistical uncertainty.

FIG. 7 shows one block 700 that is formed by segmenting and concatenating the first in-phase signal 422(1), first quadrature signal 424(1), second in-phase signal 422(2), and second quadrature signal 424(2) of FIG. 4. This segmentation may be performed by the signal processor 414 of FIG. 7 as part of calibration-free wavelength modulation spectroscopy. The signal processor 414 may also process the block 700 into a harmonic-ratio time series H that is one example of the measured spectrum 102. Accordingly, the time series H may be used as the measured-waveform array M that is processed by the method 500 of FIG. 5.

The signals 422(1), 424(1), 422(2), and 424(2) are assumed to be digitized discrete-time signals of length n (e.g., 128 or 256 points). The length n, which determines the temporal duration of the block 700, may be selected to equal one period of the ramp signal 234 (e.g., when the ramp signal 234 is a sawtooth wave) or one-half of the period of the ramp signal 234 (e.g., when the ramp signal 234 is a triangle wave). As shown in FIG. 7, the block 700 has four time series, which are denoted X₁ and Y₁ for the first in-phase signal 422(1) and first quadrature signal 424(1), respectively, and X₂ and Y₂ for the second in-phase signal 422(2) and second quadrature signal 424(2), respectively.

Mathematically, the harmonic-ratio time series H may be defined by

$$H [ i ] = \frac{{{X}_{1} [ i ]}^{2} + {{Y}_{1} [ i ]}^{2}}{{{X}_{2} [ i ]}^{2} + {{Y}_{2} [ i ]}^{2}} , \qquad ( 1 )$$

where the notation [i] indicates the i^{th} element, or sample, of the corresponding time series. As can be seen from Eqn. 1, the elements X₁[i] and Y₁[i] are squared and added to obtain a first amplitude-squared value. Similarly, the elements X₂[i] and Y₂[i] are squared and added to obtain a second amplitude-squared value. The first amplitude-squared value is then divided by the second amplitude-squared value, thereby normalizing the first amplitude-squared value with the second amplitude-squared value. The square-root of this ratio may be taken, although this adds computational complexity.

Because of the squaring in Eqn. 1, all elements of the harmonic-ratio time series H are non-negative. This property is advantageous for hashing as it prevents cancelation that occurs when some of the elements are positive and some are negatives. Another definition of the time series H that results in non-negative elements is

$$H [ i ] = \frac{\vert {X}_{1} [ i ] \vert + \vert {Y}_{1} [ i ] \vert}{\vert {X}_{2} [ i ] \vert + \vert {Y}_{2} [ i ] \vert} . \qquad ( 2 )$$

As can be seen from Eqns. 1 and 2, making each of the elements X₁[i], Y₁[i], X₂[i], and Y₂[i] non-negative (e.g., by squaring or taking the absolute value) prior to adding and division is one way to ensure that all elements of H are non-negative. Another definition of H may be used without departing from the scope hereof.

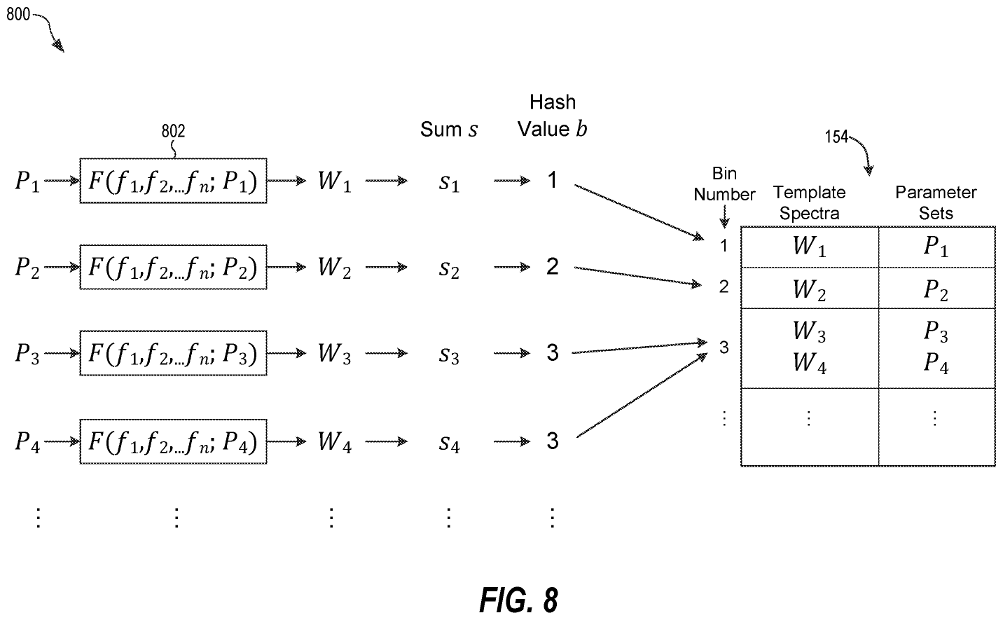

FIG. 8 illustrates a method 800 for constructing the look-up table 154. The method 800 uses a mathematical model 802 for generating the template spectra. In the example of FIG. 8, the mathematical model 802 is a function F(f; Pₖ) that takes frequency f an input. The function F may alternatively be expressed to take wavelength/as input, or another variable that relates to frequency (e.g., photon energy, wavenumber, etc.). The function F(f; Pₖ) also takes an input a parameter set Pₖ={p₁, p₂, . . . } of one or more parameters.

For each parameter p₁, p₂, . . . , a finite number of values of the parameter are sampled over a range of interest associate with that parameter. For example, if the parameter is concentration of a particular species of gas, the range of interest may range from zero to a maximum concentration to be detected by the spectroscopic gas detector 100. The number of values in the range of interest, and their spacing, may be determined by a target resolution of the gas detector 100. A superset of parameter sets is then constructed by adding to the superset one parameter set Pₖ formed from each unique combination of the sampled values of all the one or more parameters. Here, k indexes the parameter sets in the superset.

For each parameter set Pₖ in the superset, the mathematical model 802 is used to construct one corresponding template spectrum Wₖ by evaluating the model 802 at the same n frequencies f₁, f₂, . . . fₙ used for the measured spectrum 102. The model 802 also uses the one parameter set Pₖ for all n evaluations. The model 802 returns n predicted spectroscopic values {α₁*, α₂* . . . , αₓ*} in one-to-one correspondence with the n frequencies f₁, f₂, . . . fₙ, where the superscript * indicates that the spectroscopic value is the result of a mathematical calculation (as opposed to a measurement). The n predicted spectroscopic values {α₁*, α₂* . . . , αₓ*} are then stored in an array Wₖ in the same order that the measured spectroscopic values {α₁, α₂ . . . , αₙ} are stored in the measured-spectrum array M, i.e., like-numbered elements of the two arrays Wₖ and M correspond to the same frequency.

Each template spectrum Wₖ is then hashed using the same locality-sensitive hashing described above (e.g., see step 504 in FIG. 5). As shown in FIG. 8, this locality-sensitive hashing may be performed by summing the elements of the template spectrum Wₖ to generate a corresponding sum sₖ and truncating one or more of the least-significant digits of the sum sₖ to obtain an integer hash value b. The template Wₖ is then inserted into the b^{th} bin of the look-up table 154 along with the corresponding parameter set Pₖ. In some cases, not every parameter in the parameter set Pₖ needs to be stored in the look-up table 154. For example, one or more of these parameters may not be needed to determine the one or more properties 108. In this case, these parameters may be discarded to reduce the size of the look-up table 154.

In FIG. 8, the first template spectrum W₁ has a hash value b=1, and is therefore stored with the first parameter set P₁ in the first bin of the look-up table 154. Similarly, the second template spectrum W₂ has a hash value b=2, and is therefore stored with the second parameter set P₂ in the second bin of the look-up table 154. The third template spectrum W₃ and fourth template spectrum W₄ both have the same hash value b=3, and therefore both of these spectra are stored in the third bin of the look-up table 154, along with the third parameter set P₃ and fourth parameter set P₄. While FIG. 8 shows only first four parameter sets being processed and added to the look-up table 154, it should be understood that the method 800 continues over all of the parameter sets in the superset, which may number in the hundreds of thousands, or more. Accordingly, each bin of the look-up table 154 may store several template spectra and corresponding parameter sets.

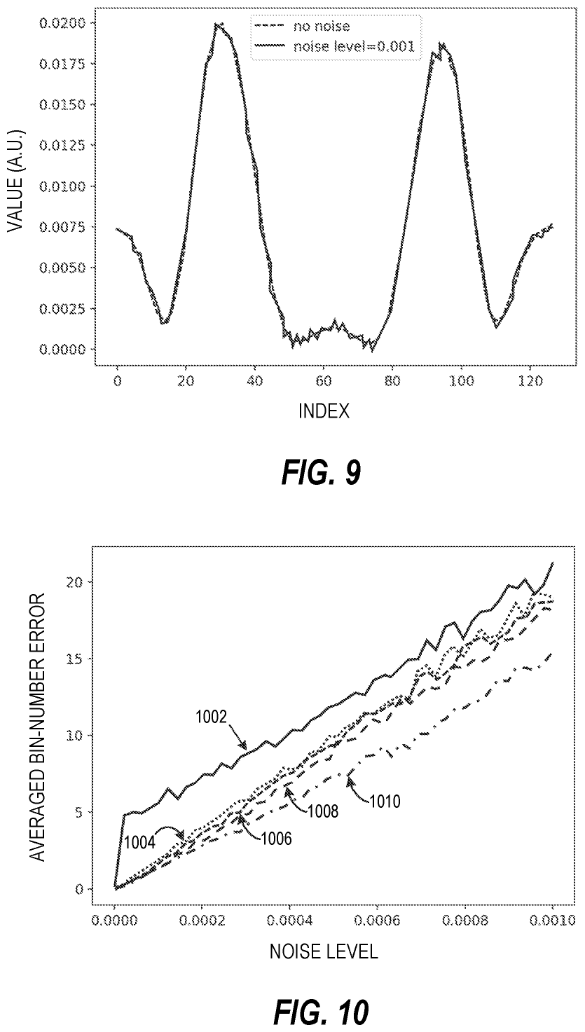

FIGS. 9 and 10 are plots that were generated from simulations that explored how the accuracy and speed of the spectroscopic gas sensor 100 depends on bin size (i.e., the number of template spectra stored in each bin) of the look-up table 154. Specifically, FIG. 9 shows a theoretical noise-free spectrum (dashed line) of 128 elements and a noisy spectrum (solid line) generated by adding noise to the noise-free spectrum. The noise-free spectrum is one of the template spectra stored in the look-up table, and therefore was generated with the mathematical model 802 of FIG. 8 for a particular parameter set. Noise was added to this noise-free spectrum by randomly sampling, for each element, a Gaussian distribution (i.e., white noise) having a width (e.g., standard deviation) of σ=0.001.

Step 504 of the method 500 (see FIG. 5) was performed with the noisy spectrum to obtain a “noisy” hash value that was compared to the actual bin number in which the noise-free spectrum was stored. This comparison produces an “bin-number error” equal to the absolute value of the difference between the noisy hash value and the actual bin number. This process was repeated 1000 times for 1000 different template spectra selected randomly from the look-up table. The resulting 1000 values of the bin-number error were averaged to produce an averaged bin-number error for the noise level. This process was repeated for several different values of the noise level and bin size.

FIG. 10 is a plot showing the averaged bin-number error as a function of the noise level for bin sizes of 2 (solid line 1002), 4 (dotted line 1004), 11 (short dashed line 1006), 22 (long dashed line 1008), and 110 (dashed-dotted line 1010). For all bin sizes except 2, the correct bin number is identified for noise levels less than ˜0.00005. The averaged bin-number is reduced as the bin size increases. Although not shown in FIG. 10, it was found that a bin size of 440 did not reduce the averaged bin-number error obtained for a bin size of 110. Accordingly, the averaged bin-number error “saturates” as bin size increases.

On the other hand, it is advantageous to reduce bin size, which speeds up operation of the spectroscopic gas sensor 100 by reducing the number of candidate spectra that must be processed to find the best-fit spectrum. For example, reducing the bin size by a factor of five from 110 to 22 will reduce the amount of time needed to process the candidate spectra by the same factor of five. However, making the bin size too small increases the probability of getting the wrong bin number. Accordingly, there is a trade-off between speed and accuracy.

It is expected that relatively large bin sizes are needed when one or more of the parameters have little impact on the sum s. One such parameter is the line center, i.e., the frequency of the center of the absorption feature. The position of the line center in the measured spectrum 102 can vary as the laser frequency f_{L} drifts. Consider template spectra that have different values of the line center but are otherwise the same. All of these template spectra will hash to similar bin numbers since changes in line center have little impact on the sum s. Accordingly, locality-sensitive hashing may not be as effective at finding the one template spectrum whose line center is closest to that of the measured spectrum 102. Instead, the template spectra may all be added to the set of candidate spectra (see the step 506 in FIG. 5), from which the best-fit spectrum can be found (see the step 508 in FIG. 5).

### Combinations of Features

Features described above as well as those claimed below may be combined in various ways without departing from the scope hereof. The following examples illustrate possible, non-limiting combinations of features and embodiments described above. It should be clear that other changes and modifications may be made to the present embodiments without departing from the spirit and scope of this invention:

(A1) A method for spectroscopic gas sensing includes operating a spectrometer to generate a measured spectrum of a gas sample and transforming, with a locality-sensitive hash function, the measured spectrum into an integer hash value h. The method also includes adding, to a candidate set of candidate spectra, template spectra stored in an h^{th} bin of a look-up table. The method also includes calculating, based on each candidate spectrum in the candidate set, a measure that quantifies discrepancy between the measured spectrum and said each candidate spectrum. The method also includes identifying, based on the measure, a best-match spectrum of the candidate spectra, and retrieving, from the h^{th} bin of the look-up table, a parameter set corresponding to the best-match spectrum. The method also includes deriving, based on the parameter set, one or more properties of the gas sample.

(A2) In the method denoted (A1), said operating the spectrometer includes scanning a frequency of a laser beam across an absorption feature of the gas sample, transmitting the laser beam through the gas sample, and photodetecting the laser beam after transmission through the gas sample.

(A3) In the method denoted (A2), the template spectra identically have a template length corresponding to a period of said scanning and the measured spectrum has a length equal to the template length.

(A4) In any of the methods denoted (A1) to (A3), said operating the spectrometer includes performing wavelength modulation spectroscopy.

(A5) In the method denoted (A4), said performing wavelength modulation spectroscopy includes performing calibration-free wavelength modulation spectroscopy.

(A6) In any of the methods denoted (A1) to (A5), said transforming includes summing elements of the measured spectrum to obtain a sum and truncating one or more least-significant digits of the sum.

(A7) In any of the methods denoted (A1) to (A6), said operating the spectrometer includes demodulating a spectroscopic signal with a local-oscillator signal to generate an in-phase signal and a quadrature signal, the local-oscillator signal having a frequency equal to a harmonic of a modulation frequency. Said operating the spectrometer also includes processing the in-phase signal and quadrature signal to generate the measured spectrum.

(A8) In the method denoted (A7), said processing includes normalizing the measured spectrum based on one or both of the in-phase signal and the quadrature signal.

(A9) In either of the methods denoted (A7) and (A8), said operating the spectrometer includes (i) frequency modulating a laser beam, prior to transmission through the gas sample, at both the modulation frequency and a ramp frequency different from the modulation frequency, (ii) transmitting the laser beam through the gas sample, and (iii) photodetecting the laser beam after transmission through the gas sample. Said demodulating occurs synchronously with said frequency modulating.

(A10) In any of the methods denoted (A1) to (A9), said operating the spectrometer includes (i) demodulating a spectroscopic signal with a first local-oscillator signal to generate a first in-phase signal and a first quadrature signal, the first local-oscillator signal having a first frequency equal to a harmonic of a modulation frequency, (ii) demodulating the spectroscopic signal with a second local-oscillator signal to generate a second in-phase signal and a second quadrature signal, the second local-oscillator signal having a second frequency equal to a harmonic of the modulation frequency, the second frequency being different from the first frequency, and (iii) processing the first in-phase signal, first quadrature signal, second in-phase signal, and second quadrature signal to generate the measured spectrum.

(A11) In the method denoted (A10), the first frequency is a first harmonic of the modulation frequency and the second frequency is a second harmonic of the modulation frequency.

(A12) In either of the methods denoted (A10) and (A11), said operating the spectrometer includes (i) frequency modulating a laser beam, prior to transmission through the gas sample, at both the modulation frequency and a ramp frequency different from the modulation frequency, (ii) transmitting the laser beam through the gas sample, and (iii) photodetecting the laser beam after transmission through the gas sample. Said demodulating the spectroscopic signal with the first local-oscillator signal and said demodulating the spectroscopic signal with the second local-oscillator signal occur synchronously with said frequency modulating.

(A13) In any of the methods denoted (A10) to (A12), each of the first in-phase signal, the first quadrature signal, the second in-phase signal, the second quadrature signal, and the measured spectrum is a sequence of n elements. Said processing includes, for each element of the sequence of n elements, (i) squaring each element of the first in-phase signal to obtain a first in-phase-squared element, (ii) squaring each element of the first quadrature signal to obtain a first quadrature-squared element, (iii) adding the first in-phase-squared element and the first quadrature-squared element to obtain a first amplitude-squared element, (iv) squaring each element of the second in-phase signal to obtain a second in-phase-squared element, (v) squaring each element of the second quadrature signal to obtain a second quadrature-squared element, (vi) adding the second in-phase-squared element and the second quadrature-squared element to obtain a second amplitude-squared element, and (vii) dividing the second amplitude-squared element by the first amplitude-squared element to obtain a corresponding element of the measured spectrum.

(A14) In any of the methods denoted (A10) to (A13), the spectroscopic signal is a digital signal. Said demodulating the spectroscopic signal with the first local-oscillator signal includes digitally multiplying the digital signal with a first digital local-oscillator waveform to generate a first digital in-phase waveform and a first digital quadrature waveform. Said demodulating the spectroscopic signal with the second local-oscillator signal includes digitally multiplying the digital signal with a second digital local-oscillator waveform to generate a second digital in-phase waveform and a second digital quadrature waveform. Said processing comprises digitally processing the first digital in-phase waveform, the first digital quadrature waveform, the second digital in-phase waveform, and the second digital quadrature waveform.

(A15) In any of the methods denoted (A1) to (A14), said calculating the measure includes calculating a residual sum of squares.

(A16) In any of the methods denoted (A1) to (A15), the method further includes storing the look-up table in a memory, the h^{th} bin being one a plurality of bins of the look-up table, each of the plurality of bins storing one or more template spectra and a parameter set corresponding to each of the one or more template spectra.

(A17) In any of the methods denoted (A1) to (A16), said adding includes adding, to the candidate set, template spectra stored in one or both of an (h-1)^{th} bin of the look-up table and an (h+1)^{th} bin of the look-up table.

(A18) In any of the methods denoted (A1) to (A17), the method further includes outputting the one or more properties of the gas sample.

(A19) In the method denoted (A18), said outputting includes outputting one or more of a temperature, a pressure, a velocity, and a concentration of a gas species.

(A20) In either of the methods denoted (A18) and (A19), said outputting includes one or both of (i) displaying the one or more properties on a screen and (ii) transmitting the one or more properties to a computing device.

(A21) In any of the methods denoted (A1) to (A20), said deriving includes outputting one or more parameters of the parameter set as the one or more properties of the gas sample.

(B1) A spectroscopic gas sensor comprising a signal processor configured to perform the method of any one or more of the methods denoted (A1) to (A21).

(B2) In the spectroscopic gas sensor denoted (B1), the signal processor includes a microprocessor core, a field-programmable gate array, and a memory in electronic communication with the microprocessor core and the field-programmable gate array, the memory storing the look-up table.

(B3) In either of the spectroscopic gas sensors denoted (B1) and (B2), the spectroscopic gas sensor further includes a photodetector configured to detect a laser beam transmitted through the gas sample.

(B4) In any of the spectroscopic gas sensors denoted (B1) to (B3), the spectroscopic gas sensor further includes a laser.

(B5) In any of the spectroscopic gas sensors denoted (B1) to (B4), the signal processor is further configured to output the one or more properties of the gas sample.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for spectroscopic gas sensing, comprising:
operating a spectrometer to generate a measured spectrum of a gas sample;
transforming, with a locality-sensitive hash function, the measured spectrum into an integer hash value h;
adding, to a candidate set of candidate spectra, template spectra stored in an h^{th} bin of a look-up table;
calculating, based on each candidate spectrum in the candidate set, a measure that quantifies discrepancy between the measured spectrum and said each candidate spectrum;
identifying, based on the measure, a best-match spectrum of the candidate spectra;
retrieving, from the h^{th} bin of the look-up table, a parameter set corresponding to the best-match spectrum; and
deriving, based on the parameter set, one or more properties of the gas sample.

2. The method of claim 1, wherein said operating the spectrometer comprises:
scanning a frequency of a laser beam across an absorption feature of the gas sample;
transmitting the laser beam through the gas sample; and
photodetecting the laser beam after transmission through the gas sample.

3. The method of claim 2, wherein:
the template spectra identically have a template length corresponding to a period of said scanning; and
the measured spectrum has a length equal to the template length.

4. The method of claim 1, wherein said operating the spectrometer comprises performing wavelength modulation spectroscopy.

5. The method of claim 4, wherein said performing wavelength modulation spectroscopy comprises performing calibration-free wavelength modulation spectroscopy.

6. The method of claim 1, wherein said transforming comprises:
summing elements of the measured spectrum to obtain a sum; and
truncating one or more least-significant digits of the sum.

7. The method of claim 1, wherein said operating the spectrometer comprises:
demodulating a spectroscopic signal with a local-oscillator signal to generate an in-phase signal and a quadrature signal, the local-oscillator signal having a frequency equal to a harmonic of a modulation frequency; and
processing the in-phase signal and quadrature signal to generate the measured spectrum.

8. The method of claim 7, wherein said processing comprises normalizing the measured spectrum based on one or both of the in-phase signal and the quadrature signal.

9. The method of claim 7, wherein:
said operating the spectrometer comprises:
frequency modulating a laser beam, prior to transmission through the gas sample, at both the modulation frequency and a ramp frequency different from the modulation frequency;
transmitting the laser beam through the gas sample; and
photodetecting the laser beam after transmission through the gas sample; and
said demodulating occurs synchronously with said frequency modulating.

10. The method of claim 1, wherein said operating the spectrometer comprises:
demodulating a spectroscopic signal with a first local-oscillator signal to generate a first in-phase signal and a first quadrature signal, the first local-oscillator signal having a first frequency equal to a harmonic of a modulation frequency;
demodulating the spectroscopic signal with a second local-oscillator signal to generate a second in-phase signal and a second quadrature signal, the second local-oscillator signal having a second frequency equal to a harmonic of the modulation frequency, the second frequency being different from the first frequency; and
processing the first in-phase signal, first quadrature signal, second in-phase signal, and second quadrature signal to generate the measured spectrum.

11. The method of claim 10, wherein:
the first frequency is a first harmonic of the modulation frequency; and
the second frequency is a second harmonic of the modulation frequency.

12. The method of claim 10, wherein:
said operating the spectrometer comprises:
frequency modulating a laser beam, prior to transmission through the gas sample, at both the modulation frequency and a ramp frequency different from the modulation frequency;
transmitting the laser beam through the gas sample; and
photodetecting the laser beam after transmission through the gas sample; and
said demodulating the spectroscopic signal with the first local-oscillator signal and said demodulating the spectroscopic signal with the second local-oscillator signal occur synchronously with said frequency modulating.

13. The method of claim 10, wherein:
each of the first in-phase signal, the first quadrature signal, the second in-phase signal, the second quadrature signal, and the measured spectrum comprises a sequence of n elements;
said processing comprises, for each element of the sequence of n elements:
squaring each element of the first in-phase signal to obtain a first in-phase-squared element;
squaring each element of the first quadrature signal to obtain a first quadrature-squared element;
adding the first in-phase-squared element and the first quadrature-squared element to obtain a first amplitude-squared element;
squaring each element of the second in-phase signal to obtain a second in-phase-squared element;
squaring each element of the second quadrature signal to obtain a second quadrature-squared element;
adding the second in-phase-squared element and the second quadrature-squared element to obtain a second amplitude-squared element; and
dividing the second amplitude-squared element by the first amplitude-squared element to obtain a corresponding element of the measured spectrum.

14. The method of claim 10, wherein:
the spectroscopic signal is a digital signal;
said demodulating the spectroscopic signal with the first local-oscillator signal comprises digitally multiplying the digital signal with a first digital local-oscillator waveform to generate a first digital in-phase waveform and a first digital quadrature waveform;
said demodulating the spectroscopic signal with the second local-oscillator signal comprises digitally multiplying the digital signal with a second digital local-oscillator waveform to generate a second digital in-phase waveform and a second digital quadrature waveform; and
said processing comprises digitally processing the first digital in-phase waveform, the first digital quadrature waveform, the second digital in-phase waveform, and the second digital quadrature waveform.

15. The method of claim 1, wherein said calculating the measure comprises calculating a residual sum of squares.

16. The method of claim 1, further comprising storing the look-up table in a memory, the h^{th} bin being one a plurality of bins of the look-up table, each of the plurality of bins storing one or more template spectra and a parameter set corresponding to each of the one or more template spectra.

17. The method of claim 1, wherein said adding comprises adding, to the candidate set, template spectra stored in one or both of an (h−1)^{th} bin of the look-up table and an (h+1)^{th} bin of the look-up table.

18. The method of claim 1, further comprising outputting the one or more properties of the gas sample.

19. The method of claim 18, wherein said outputting comprises outputting one or more of a temperature, a pressure, a velocity, and a concentration of a gas species.

20. The method of claim 18, wherein said outputting comprises one or both of:
displaying the one or more properties on a screen; and
transmitting the one or more properties to a computing device.

21. The method of claim 1, wherein said deriving comprises outputting one or more parameters of the parameter set as the one or more properties of the gas sample.

22. A spectroscopic gas sensor comprising a signal processor configured to perform the method of claim 1.

23. The spectroscopic gas sensor of claim 22, the signal processor comprising:
a microprocessor core;
a field-programmable gate array; and
a memory in electronic communication with the microprocessor core and the field-programmable gate array, the memory storing the look-up table.

24. The spectroscopic gas sensor of claim 22, further comprising a photodetector configured to detect a laser beam transmitted through the gas sample.

25. The spectroscopic gas sensor of claim 24, further comprising a laser configured to generate the laser beam.

26. The spectroscopic gas sensor of claim 22, the signal processor being further configured to output the one or more properties of the gas sample.
